(19) United States
(12) Patent Application Publication
(10) Pub. No.: US 2002/0032350 A1
(43) Pub. Date: Mar. 14, 2002

(54) Process for reprocessing mass streams containing mesityl oxide

(75) Inventors: Otto Gerlich, Gladbeck (DE); Heinrich Van Barneveld, Bottrop (DE); Wilfried Jordon, Dorsten (DE)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: PHENOLCHEMIE GMBH & CO. KG, GLADBECK (DE)

(21) Appl. No.: 09/951,755

(22) Filed: Sep. 14, 2001

(30) Foreign Application Priority Data
Sep. 14, 2000 (DE) 10 045 355.4

(51) Int. Cl. C07C 45/00

(52) U.S. Cl. 568/383

(57) ABSTRACT

A mass stream comprising mesityl oxide may be reacted with water in an acidic medium, a basic medium, or in the presence of an acidic catalyst, to convert the mesityl oxide to acetone. This process may be used in conjunction with the Hock process for manufacturing phenol and acetone to decrease the amount of mesityl oxide by-product, and increase the yield of acetone. Thus, cumene is oxidized to form cumene hydroperoxide, which is then cleaved in the presence of an acidic catalyst to form a cleavage product phase comprising phenol, acetone, and mesityl oxide. The cleavage product phase may then be distilled to provide a mass stream comprising mesityl oxide. At least some of the mesityl oxide of the mass stream is then, according to the process of the present invention, reacted with water, in an acidic medium, a basic medium, or in the presence of an acidic catalyst, thereby producing acetone.

### BACKGROUND OF THE INVENTION

Field of the Invention

The present invention relates to a process for reprocessing mass streams containing mesityl oxide, particularly a process for recovering acetone from mass streams containing mesityl oxide produced during the acid-catalyzed cleavage of cumene hydroperoxide, by means of an acidic or alkaline cleavage of mesityl oxide.

In the conventional and economically important Hock process for recovering phenol and acetone from mass streams containing cumene, large quantities of acetone are produced during the acid-catalyzed cleavage of cumene hydroperoxide. Often the undesired by-products are removed from the resulting acetone by treating it with alkaline reagents. Under these acidic or alkaline reaction conditions, the acetone reacts with itself in a first step to form diacetone alcohol, which can then form mesityl oxide (i.e., 4-methy-3-penten-2-one) or decompose back into acetone again when water is added. The mesityl oxide that is produced causes a yield loss of acetone, which affects the operational efficiency of the manufacture of phenol and acetone using the Hock process.

During the manufacture of phenol by the acid-catalyzed cleavage of cumene hydroperoxide, other by-products and/or contaminants are formed, such as α-methyl styrene, cumene and other compounds that are present in the cleavage product phase, besides phenol and acetone. These by-products and contaminants must then be separated from the phenol and the acetone. This separation is usually carried out by means of extraction and/or distillation processes. Distilling the cleavage product phase also produces fractions containing, among other things, mesityl oxide, in addition to fractions mainly containing phenol or acetone. Distilling the fractions containing mesityl oxide provides new fractions that, in some cases, contain more than 50% by weight of mesityl oxide. However, it has not been economically feasible to utilize such mesityl oxide containing fractions, for example to make useful compounds such as methyl isobutyl ketone. As a result, such mesityl oxide containing fractions have generally been incinerated to provide heat energy in the phenol process. Thus, an economically viable process for reprocessing mass streams containing mesityl oxide has been needed.

Surprisingly, an economically viable process for recovering acetone from mesityl, oxide has been discovered, which utilizes the reverse of the formation reaction of mesityl oxide from acetone. Thus, acetone may be economically recovered from mass streams containing mesityl oxide and thereby reduce the yield loss of acetone in the overall phenol manufacturing process.

### SUMMARY OF THE INVENTION

Accordingly, it is an object of the present of the present invention to provide a method of reprocessing mixtures containing mesityl oxide, by adding water to mixtures containing mesityl oxide in an acidic or basic medium, or by reacting mesityl oxide with water in the presence of an acidic catalyst, thereby cleaving at least part of the mesityl oxide into acetone.

It is another object of the present invention to provide a process for manufacturing phenol and acetone from cumene according to the Hock process, comprising first forming cumene hydroperoxide by oxidizing cumene, cleaving the cumene hydroperoxide under acid-catalyzed conditions, distilling the cleavage product phase, and converting at least some of the mesityl oxide into acetone by adding water to the mesityl oxide containing phase in an acidic or basic medium, or by adding water to mesityl oxide in the presence of an acidic catalyst.

### BRIEF DESCRIPTION OF DRAWINGS

FIG. 1 is a diagram of an apparatus for carrying out an embodiment of the process of the present invention.

### DETAILED DESCRIPTION OF THE INVENTION

Mass streams containing mesityl oxide, which are not suitable as a starting product for the manufacture of derivatives such as methyl isobutyl ketone, can be economically reprocessed using the process of the present invention. At least part of the mesityl oxide can be converted into acetone by causing it to react with water. The acetone may then be separated from the reaction mixture relatively easily, for example by distillation, and may thereby be available for further processing or further utilization.

A further advantage of the process of the present invention is that the operational efficiency of the Hock process for manufacturing phenol may be increased considerably. Using the process of the present invention, the quantity of mesityl oxide by-product provided by the Hock process may be reduced, thereby reducing the yield loss of acetone below that of conventional processes.

Two molecules of acetone react under acidic or alkaline conditions, to form diacetone alcohol, followed by dehydration of the diacetone alcohol to form mesityl oxide. The method of the present invention reverses these reactions in order to reduce the amount of mesityl oxide in mass streams containing mesityl oxide. Thus, water, or an acidic or alkaline medium containing water, is added to a mass stream containing mesityl oxide, and then thermally treated in an acidic or alkaline environment. If only water is added to the mass stream containing mesityl oxide, the thermal treatment must be carried out in the presence of an acidic catalyst. An acidic catalyst may be, for example, phyllosilicates, zeolites or acidic ion-exchange resins. Examples of suitable zeolites include natural zeolites, such as chabazite, mordenite, erionite, faujasite, and clinoptilolite, and synthetic zeolites such as zeolite A, zeolite X, zeolite Y, zeolite L, zeolite omega, zeolite H, and ZSM-5. Acidic ion-exchange resins include sulfonated resins, such as sulfonated polystyrene or sulfonated fluorocarbon resins. Preferably, a mineral acid such as hydrochloric acid or sulfuric acid, or an organic acid such as acetic acid is added as an acidic medium to the mass stream containing mesityl oxide. Particularly preferably, the acidic medium is diluted sulfuric acid, having a sulfuric acid concentration of 0.1 to 25 wt. %, preferably 1 to 10 wt. %, most preferably 1 to 5 wt. % . The basic medium may be, for example, an alkaline lye such as a sodium hydroxide solution or a potassium hydroxide solution, an alkaline-earth lye, such as solutions of magnesium or calcium hydroxide, or also organic phases comprising compounds with basic properties, such as amines or phenolates. Most particularly preferably, a 1 to 10 wt. %, preferably an approximately 4 wt. % sodium hydroxide solution may added to the mesityl oxide containing mass stream that is to be treated. Preferably, sufficient acidic or basic medium is added to the mass stream containing mesityl oxide so that the reaction mixture consists of a mass stream containing mesityl oxide and an acidic or basic medium comprising 5 to 15% of the volume, more preferably 5 to 10% by volume in relation to the total volume of the reaction mixture.

The reaction mixture, which should be well mixed, may be thermally treated by heating it to a temperature of 60 to 95° C., preferably to a temperature of 80 to 90° C. When it is heated, the mesityl oxide that is present in the reaction mixture reacts to form diacetone alcohol under the acidic or basic conditions of the medium that is used, or with water in the presence of the acidic catalyst. The diacetone alcohol then decomposes into two acetone molecules. The acetone may be removed from the reaction mixture by known processes, such as distillation.

The process of the present invention may be carried out as a continuous process, or as a discontinuous (i.e., batch process), preferably as a discontinuous process. Water may be added during the process of the present invention to replace the water that is consumed in the reaction. A mass stream containing mesityl oxide may also be added to the reaction mixture so that the mesityl oxide that has been converted to diacetone alcohol or acetone is replaced.

The process of the present invention may be used in virtually any mass stream containing mesityl oxide. Preferably, the mass stream containing mesityl oxide should contain more than 20% by weight of mesityl oxide, particularly preferably from 30 to 90% by weight of mesityl oxide and most particularly preferably between 50 and 85% by weight of mesityl oxide. Advantageously, the mass stream may be enriched in mesityl oxide, e.g. by distillation or other suitable unit processes.

The process of the present invention may be exemplified by reprocessing a cleavage product phase of a Hock process, by means of the acid-catalyzed cleavage of cumene hydroperoxide. However, as discussed above, the process of the present invention is not limited to this embodiment.

In the Hock process, cumene hydroperoxide is produced by reacting cumene and oxygen. The cumene hydroperoxide produced is then cleaved to phenol and acetone under acid-catalyzed conditions. However, in addition to the cleavage reaction, secondary reactions also produce by-products, so that the cleavage product phase also contains other compounds, in addition to the phenol, acetone and catalyst.

Generally, the organic part of this cleavage product phase is reprocessed by distillation and/or extraction to separate the various constituents of this phase. Separation by distillation is usually carried out in several distillation columns connected in series. Because they have similar boiling temperatures or chemical attractive powers, the distillation fractions obtained often comprise several compounds. These fractions are therefore often reprocessed further, generally in further distillation stages.

In the process of the present invention, mass streams, such as distillation fractions containing mesityl oxide, and mass streams from the direct reprocessing of the cleavage product phase, especially from reprocessing acetone, but also from the side streams or fractions that can result from the reprocessing or recovery of byproducts, are combined and reprocessed by distillation in such a way that a fraction or a flux is obtained which contains at least 20% by weight of mesityl oxide, preferably from 30 to 90%, in particular preferably from 50 to 85%. This process can be carried out as a continuous or a discontinuous process, preferably a continuous process. The fractions thus obtained, and also smaller phases that contain at least 20% by weight of mesityl oxide, preferably from 30 to 90%, in particular preferably from 50 to 85%, are reacted with water in an acidic or basic medium, or in the presence of an acidic catalyst, by thermally treating the mixture. This causes water to react with the mesityl oxide, thus forming diacetone alcohol. The diacetone alcohol then cleaves into two molecules of acetone, so that two moles of acetone are formed for each mole of converted mesityl oxide. The thermal treatment and the reaction should preferably take place at a temperature of 80 to 90° C. and a pressure of 800 to 1100 mbar.

The acidic or basic media may be acids or bases or solutions containing acids or bases. Preferred acids are inorganic mineral acids such as hydrochloric or sulfuric acid or water-soluble organic acids such as acetic acid. The most particularly preferred acidic medium is diluted sulfuric acid, at a concentration of 0.1 to 25 wt. %, preferably 1 to 10 wt. %, most preferably 1 to 5 wt. %. Bases may include organic or inorganic bases such as organic amines, ammonia or alkali lye or alkaline earth lye.

The most preferred basic medium is a sodium hydroxide solution, preferably a 1 to 10% sodium hydroxide solution and particularly preferably an approximately 4% sodium hydroxide solution. Preferably the acidic media have a pH of 1 to 3.

Preferably the basic media have a pH of 8 to 10. The water required for the reaction can come from the water in the acidic or basic medium, or it can be added separately to the reaction mixture. Acidic catalysts may be zeolites or acidic ion exchangers. If such catalysts are used, at least one mole of water must be added to the reaction mixture for every mole of mesityl oxide that is to be cleaved, preferably an excess of water, for example, a molar excess of 1 to 100%, preferably 10 to 50%.

The acidic or basic media are brought into intimate contact with the mass stream containing mesityl oxide that is to be treated. As this mass stream often contains other organic compounds besides mesityl oxide, which may not be very soluble or may be completely insoluble in water, good mixing is required to produce the intimate contact between the mass stream and the acidic or basic medium. Mixing may be carried out using known methods. Preferably, the acidic or basic media and the mass stream containing mesityl oxide are mixed using pumps, agitators or, in the case of thermosiphon circulation, in an evaporator.

The reaction mixture, comprising the mass stream containing mesityl oxide and an acidic or basic medium, is heated or thermally treated during or after the mixing process, preferably during the mixing process. Mixing and heating can take place in one or in several pieces of apparatus, preferably in one piece of apparatus. The apparatus can be, for example, a reactor, a reaction column, a distillation column, a mixing vessel, or similar devices. The preferred apparatus is a distillation column or a reaction column, particularly preferably, a still. The reaction of water with mesityl oxide may be carried out in this apparatus at temperatures of 80 to 90° C., and a pressure of 800 to 1100 mbar.

Because water is consumed during the reaction, water is preferably added to the apparatus in which the reaction takes place, for example in the reaction vessel or the reaction or distillation column or still, to replace the water that has been consumed. Preferably, sufficient water is replaced so that the concentration of the lye in the mixture is maintained at approximately 4%. In addition, it is preferred that a sufficient mass stream or fraction containing mesityl oxide is added to the mixture to replace the mesityl oxide that has been converted to acetone.

The acetone that is produced from the reaction of mesityl oxide with water can be separated from the other components of the mixture by various known methods. Preferably the acetone is separated from the mixture by distillation. The reaction mixture can either be fed to a distillation column and be separated by distillation or the reaction itself can take place in a distillation column or still, so that acetone forming during the reaction can be distilled off from the reaction mixture.

In all cases it is preferable that the acetone be discharged at the top of the distillation column or still in a gaseous form. Advantageously, part of the acetone that has been discharged may be condensed and fed back to the column as a reflux. In this way one can avoid discharging large quantities of undesired components from the column together with the acetone. The crude acetone obtained in this manner may be further reprocessed. Preferably the crude acetone obtained by the process of the present invention may be combined with the acetone stream produced during the reprocessing of the cleavage product phase. These combined acetone phases are then reprocessed together.

The mixture remaining in the bottom of the distillation column or still, which can contain unreacted mesityl oxide, cumene and other products and/or by-products from the phenol manufacturing process, may be either reprocessed or incinerated. Reprocessing is preferably carried out by removing the mixture from the bottom of the distillation column or still, and separating it by a known method into an organic and an aqueous phase. The aqueous phase, which mainly contains the acidic or basic medium used in the reaction can be fed back to the process for reprocessing mesityl oxide. Depending on the composition, the organic phase, which may contain mesityl oxide, cumene and organic products, by-products or educts from the phenol manufacturing process, is either thermally reprocessed or fed back to the process for reprocessing the cleavage product phase, to recover any cumene that is present.

Conversion of the phase containing mesityl oxide with water in an acidic or organic medium and the subsequent distillation can be carried out as a discontinuous process or as a continuous process, preferably as a discontinuous process.

The reaction of mesityl oxide with water and the subsequent purification of the resulting acetone by distillation may be carried out in separate pieces of apparatus, e.g. in a reaction vessel and a separate distillation column or still, or in one single piece of apparatus, preferably in one single piece of apparatus, most preferably in a distillation column or still.

In a special embodiment of the process of the present invention, acidic catalysts may be used to carry out the reaction of mesityl oxide and water, instead of an acidic or basic medium. Suitable acidic catalysts include acidic ion-exchange resins or zeolites or phyllosilicates, especially phyllosilicates or H form zeolites. In this embodiment of the process of the present invention, water and a mass stream containing mesityl oxide are mixed, heated, and brought into contact with the catalyst. It is particularly important, as discussed above, that the water and the mass stream containing mesityl oxide are well mixed. Mixing may be carried out in a conventional manner, e.g. as described above. Again, the reaction and the subsequent distillation process may be carried out in one single piece of apparatus such as a still.

Depending on the temperature stability of the catalyst that is used, it may be advantageous to carry out the reaction and the subsequent distillation process in two separate vessels. For example, the well mixed reaction mixture of water and the phase containing mesityl oxide may be heated to a temperature of 60 to 80° C. and fed to a piece of apparatus, e.g. an ion exchanger filled with an acidic ion-exchange resin catalyst, such as Levatit SPC 108. The reaction mixture that is treated in the apparatus or ion exchanger may then be fed into a distillation column to separate the acetone from the rest of the reaction mixture, as described above. Part of the mixture being treated in the ion exchanger and/or part of the residual reaction mixture in the bottom of the distillation column may be fed back into the ion exchanger. The apparatus containing the catalyst may be a fixed-bed or fluidized-bed reactor or similar apparatus, instead of an ion exchanger. This embodiment of the process of the present invention may also be carried out as a continuous process or as a discontinuous process, preferably a discontinuous process.

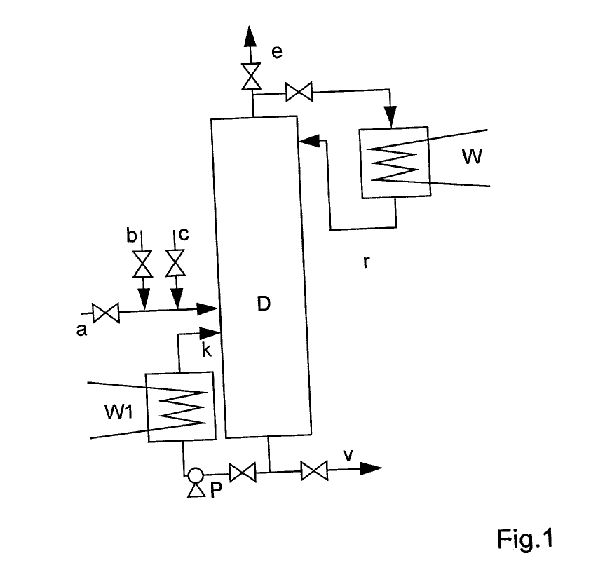

FIG. 1 shows an example of an embodiment of the process according to the present invention, without limiting the process to this example. The phase to be treated containing mesityl oxide is fed to the distillation column “D” via pipe “a”.

Lye or acid may be added to the mesityl oxide containing phase via pipe “b”, and water may be added to the mesityl oxide phase via pipe “c”. The mixture, consisting of the mesityl oxide containing phase, lye or acid, and water, is heated in column “D.” The acetone that is produced from the reaction of mesityl oxide with water is steam distilled off via the column head, whereby it is either fed directly to a further reprocessing stage via pipe “a,” or at least partially condensed in heat exchanger “W”, and returned to the column “D” via pipe “r.” The less volatile constituents, such as unreacted mesityl oxide, or other by-products or contaminants from the phenol production or acetone reprocessing contained in the mesityl oxide containing phase, such as cumene or lye, acid and/or water, are concentrated in the bottom of the column. This mixture is fed back to the distillation column by means of pump “P,” via pipe “k” and heat exchanger “W1.” Part of the mixture removed from the bottom can be fed to an additional reprocessing stage or incineration via pipe “v” after phase separation.

The process of the present invention may be explained in more detail by way of the following example without being limited to this example.

### EXAMPLE 1

78.8 tons of a ketone fraction containing 51% mesityl oxide were added to a still. A sufficient quantity of a 4% sodium hydroxide solution was added to the ketone fraction so that a mixture was formed containing approximately 10% by volume of sodium hydroxide solution. The components were brought into intimate contact by mixing with a pump, and the mixture was heated to a temperature of approximately 85° C. The acetone that was produced was distilled off at the head of the still, whereby part of the distilled acetone was returned to the still as reflux.

As water was consumed during the reaction, a constant amount of 0.5tons/hour of water was added, so that the concentration of the lye was maintained at a value of approximately 4%. At the same time, 2.5 tons/hour of the ketone fraction was added to replace the converted mesityl oxide.

An analysis of the process of this example showed that approximately 75% of the 51% of mesityl oxide contained in the 78.8 ton ketone fraction were converted to acetone, which corresponds to a 39.5 tons of acetone. This example illustrates that the quantity of a ketone fraction that can only be used for combustion can be considerably reduced by treating the ketone fraction by the process of the present invention.

The priority document of the present application, German application 100 45 355.4, filed Sep. 14, 2000, is incorporated herein by reference.

Obviously, numerous modifications and variations on the present invention are possible in light of the above teachings. It is therefore to be understood that within the scope of the appended claims, the invention may be practiced otherwise than as specifically described herein.

WHAT IS CLAIMED AS NEW AND IS INTENDED TO BE SECURED By letters patent is:

1. A process for treating a mass stream comprising mesityl oxide, comprising:
reacting mesityl oxide with water in an acidic medium, a basic medium, or in the presence of an acidic catalyst, thereby producing acetone.

2. A process for manufacturing phenol and acetone comprising:
oxidizing cumene to form cumene hydroperoxide;
cleaving the cumene hydroperoxide in the presence of an acidic catalyst to form a cleavage product phase comprising phenol, acetone, and mesityl oxide;
distilling the cleavage product phase to provide a mass stream comprising mesityl oxide; and
reacting at least some of the mesityl oxide of the mass stream with water in an acidic medium, a basic medium, or in the presence of an acidic catalyst, thereby producing acetone.

3. The process of claim 1, wherein the mass stream is distilled to concentrate the mesityl oxide.

4. The process of claim 2, wherein the mass stream is distilled to concentrate the mesityl oxide.

5. The process of claim 1, wherein the mass stream comprises at least 20% by weight of mesityl oxide.

6. The process of claim 2, wherein the mass stream comprises at least 20% by weight of mesityl oxide.

7. The process of claim 1, wherein the mass stream comprises 50 to 85% by weight of mesityl oxide.

8. The process of claim 2, wherein the mass stream comprises 50 to 85% by weight of mesityl oxide.

9. The process of claim 1, wherein a basic or acidic medium is added to the mass stream comprising mesityl oxide.

10. The process of claim 2, wherein a basic or acidic medium is added to the mass stream comprising mesityl oxide.

11. The process of claim 1, wherein the acidic medium is a mineral acid or an organic acid.

12. The process of claim 2, wherein the acidic medium is a mineral acid or an organic acid.

13. The process of claim 1, wherein the basic medium is an alkali lye or an organic phase containing phenolates or amines.

14. The process of claim 2, wherein the basic medium is an alkali lye or an organic phase containing phenolates or amines.

15. The process of claim 1, wherein the basic medium is a 1 to 10% by weight sodium hydroxide solution.

16. The process of claim 2, wherein the basic medium is a 1 to 10% by weight sodium hydroxide solution.

17. The process of claim 1, wherein the mass stream comprising mesityl oxide is mixed with an acidic or basic medium to form a mixture, and the acidic or basic medium comprises 5 to 15% by volume of the total volume of the mixture.

18. The process of claim 2, wherein the mass stream comprising mesityl oxide is mixed with an acidic or basic medium to form a mixture, and the acidic or basic medium comprises 5 to 15% by volume of the total volume of the mixture.

19. The process of claim 1, wherein the mass stream comprising mesityl oxide is mixed with an acidic or basic medium to form a mixture, and the acidic or basic medium comprises approximately 110% by volume of the total volume of the mixture.

20. The process of claim 2, wherein the mass stream comprising mesityl oxide is mixed with an acidic or basic medium to form a mixture, and the acidic or basic medium comprises approximately 10% by volume of the total volume of the mixture.

21. The process of claim 1, wherein the mass stream comprising mesityl oxide is mixed with water and thermally treated in the presence of an acidic catalyst.

22. The process of claim 2, wherein the mass stream comprising mesityl oxide is mixed with water and thermally treated in the presence of an acidic catalyst.

23. The process of claim 21, wherein the acidic catalyst is a zeolite or an acidic ion-exchange resin.

24. The process of claim 22, wherein the acidic catalyst is a zeolite or an acidic ion-exchange resin.

25. The process of claim 1, wherein the reacting is carried out at a temperature of 60 to 95° C.

26. The process of claim 2, wherein the reacting is carried out at a temperature of 60 to 95° C.

27. The process of claim 25, wherein the reacting is carried out at a temperature of 80 to 90° C.

28. The process of claim 26, wherein the reacting is carried out at a temperature of 80 to 90° C.

29. The process of claim 1, further comprising removing the acetone by distillation.

30. The process of claim 2, further comprising removing the acetone by distillation.

31. The process of claim 1, wherein the reacting is carried out in one or more apparatus.

32. The process of claim 2, wherein the reacting is carried out in one or more apparatus.

33. The process of claim 1, wherein the reacting is carried out in a still.

34. The process of claim 2, wherein the reacting is carried out in a still.

35. The process of claim 29, wherein the reacting is carried out in a reactor or an ion exchanger and removing the acetone is carried out in a distillation column.

36. The process of claim 30, wherein the reacting is carried out in a reactor or an ion exchanger and removing the acetone is carried out in a distillation column.

37. The process of claim 1, wherein the reacting is carried out in an apparatus, and an amount of a phase comprising mesityl oxide and water is added to the apparatus sufficient to replace the mesityl oxide and water consumed by the reacting.

38. The process of claim 2, wherein the reacting is carried out in an apparatus, and an amount of a phase comprising mesityl oxide and water is added to the apparatus sufficient to replace the mesityl oxide and water consumed by the reacting.
